(19) United States
(12) Patent Application Publication
(10) Pub. No.: US 2012/0189674 A1
(43) Pub. Date: Jul. 26, 2012

(54) Compositions for topical delivery of the pharmacologically active compounds

(76) Inventors: Kalidas M. Kale, (US); Amruta K. Kale, (US); Samesh K. Kale, (US)

(21) Appl. No.: 13/012,238

(22) Filed: Jan. 24, 2011

(51) Int. Cl. A61K 8/11; A61P 17/00; A61P 17/10; A61P 17/06; A61Q 19/08; A61Q 17/04; A61P 29/00; A61P 17/04; A61P 17/14; A61P 31/04; A61P 31/10; A61P 33/00; A61P 31/12; A61K 36/736; A61K 9/00

(52) U.S. Cl. 424/401; 424/400; 424/59; 424/735

(57) ABSTRACT

Novel compositions for topical drug delivery of the pharmaceutically active compounds which allow active compounds to pass through the stratum corneum, epidermis and deliver to the deeper layers of the skin, dermis, are developed. The active compounds are encapsulated into the phospholipid spheres or bilayers. The phospholipid emulsion technology utilized in the skincare products provides a vastly superior method of active ingredient delivery. This method allows pharmacologically active compounds to penetrate through outer layers of the skin to reach the deep layers. As a result, the products are highly effective, cosmetically elegant and gentle on the skin.

### BACKGROUND

The stratum corneum (SC) is the rate-limiting barrier for most topically applied drug products. The SC also lies in a direct path to the viable tissues of the skin where many diseases of the skin manifest themselves. Due to the difficulty of passing through the SC most of the topical drug delivery systems are marginally effective. In order to be efficacious, topically applied drug must pass through the SC and reach the viable tissues below the site where most drugs must be delivered. Regardless of how far through the skin layers, stratum Corneum-epidermis-dermis, the drug needs to penetrate, it needs to pass through the SC first before reaching deeper skin layers.

Non-limiting examples of actives useful in compositions of the invention include anti-Eczema treatments, sunless tanning actives, skin lightening actives, anti-acne actives, anti-psoriasis actives, anti-skin wrinkling and anti-skin aging actives, vitamins, anti-inflammatory actives, anesthetic actives, analgesic actives, anti-pruritic actives, anti-microbial actives (e.g. antifungals, antibacterials, and antiparasitics), anti-virals, anti-allergenics, medicinal actives (e.g., skin rash, skin disease and dermatitis medications), anti-cellulite additives, insect repellant actives, antioxidants, hair growth promoters, hair growth inhibitors, hair bleaching agents, deodorant compounds, plant extracts and mixtures and combinations thereof.

TABLE 1
Pharmacologically Active Compounds Useful in Compositions of the Invention for
Topical Delivery

| Class | Active compounds |
|---|---|
| Plant Extracts | Aloe vera, Basil, Neem, Sandalwood, Turmeric |
| Antifungal | Antifungal actives include, but are not limited to, Terbinafine |
| | hydrochloride, Amphotericin B, Betamethasone dipropionate, |
| | Butenafine hydrochloride, Butoconazole nitrate, Caspofungin |
| | acetate, Chloroxylenol, Undecylenic acid, Ciclopirox, |
| | Clotrimazole, Econazole nitrate, Griseofulvin, Ketoconazole, |
| | Micafungin sodium, Miconazole nitrate, Naftifine hydrochloride, |
| | Nystatin, Oxiconazole nitrate, Sertaconazole nitrate, and Voriconazole. |
| Acne | Actives to treat acne include but not limited to, Benzoyl |
| | peroxide, Clindamycin phosphate, Benzoyl peroxide & |
| | Clindamycin, Benzoyl peroxide & Clindamycin phosphate, |
| | Erythromycin, isotretinoin, Sodium sulfacetamide & sulfur, |
| | Sulfacetamide sodium, 5,7-dichloro-8-hydroxyquinoline, |
| | Resorcinol, Resorcinol acetate, Salicylic acid, Azaleic acid, |
| | long chain dicarboxylic acids, Sulfur, Zinc, various natural |
| | agents such as those derived from green tree, and mixtures thereof. |
| Anti-aging Skin | Include but not limited to, alpha hydroxy acids and derivatives: |
| Renewal Actives | Ammonium glycolate, Ammonium lactate, Arginine glycolate, |
| | Citric acid, Fruit acids, Glycolic acid, Honey extract, |
| | Hyaluronic acid (glycosaminoglycon), Lactic acid, Malic acid, |
| | Pyruvic acid, Sodium glycolate, Sodium lactate, Tartaric acid, |
| | Beta Hydroxy Acids and Derivatives: including Salicylic acid |
| | and its salts. |
| | Retinoids or carotenoids including Retinoic acid, Tretinoin, |
| | Vitamin A (retinol) |
| | Biological lipid surfactants including Ceramides, Sphingoids |
| | Tricloracetic acid (TCA) |
| Skin Lighteners | Include but not limited to, Ascorbic acid, Arbutin acid, |
| | Hydroquinone, Kojic acid, and their derivatives. Paper |
| | mulberry extract, Phenylethyl resorcinol, Niacin amide |
| Steroidal Anti- | Include but not limited to, Hydrocortisone, Hydrocortisone |
| Inflammatory | acetate, Hydrocortisone butyrate |
| Actives | |
| Antioxidants | Include but not limited to, Vitamin C, Vitamin E and Coenzyme |
| | Q-10 |
| Sunscreen | Chemicals used in sunscreens to absorb UVA and UVB |
| | radiation, include but not limited to, Benzophenones, |
| | Dioxybenzone, Avobenzone, Para-amino benzoic acid (PABA), |
| | PABA esters: Padimate O, Padimate A and Glyceryl amino |
| | benzoate, Cinnamates: Octyl methoxycinnamate and Cinoxate |
| | Salicylates: homomenthyl salicylate, Octyl salicylate, |
| | Triethanolamine salicylate |
| a₂-Adrenergic | Include but not limited to Yohimbine, Piperoxan, Phentolamine, |
| Inhibitors | dihydroergotamine |
| Nonsteroidal Anti- | Non-limiting examples of non-steroidal anti-inflammatory |
| Inflammatory | agents suitable for use herein include Oxicams (e.g., Piroxicam, |
| Agents | Isoxicam, Tenoxicam, Sudoxicam, CP-14,304); salicylates |
| | (e.g., Aspirin, Disalcid, Benorylate, Trilisate, Safapryn, Solprin, |
| | Diflunisal, Fendosal); acetic acid derivatives (e.g., Diclofenac, |
| | Fenclofenac, Indomethacin, Sulindac, Tolmetin, Isoxepac, |
| | Furofenac, Tiopinac, Zidometacin, Acematacin, Fentiazac, |
| | Zomepirac, Clindanac, Oxepinac, Felbinac, Ketorolac); f |
| | Fenamates (e.g., Mefenamic, Meclofenamic, Flufenamic, |
| | Niflumic, Tolfenamic acids); Propionic acid derivatives (e,g., |
| | Ibuprofen, Naproxen, Benoxaprofen, Flurbiprofen, Ketoprofen, |
| | Fenoprofen, Fenbufen, Indopropfen, Pirprofen, Carprofen, |
| | Oxaprozin, Pranoprofen, Miroprofen, Tioxaprofen, Suprofen, |
| | Alminoprofen, Tiaprofenic); Pyrazoles (e.g., Phenylbutazone, |
| | Oxyphenbutazone, Feprazone, Azapropazone, Trimethazone); |
| | and combinations thereof |
| Hair Growth | Actives suitable for treating hair loss include, but are not |
| | limited to potassium channel openers or peripheral vasodilators |
| | such as Minoxidil, Diazoxide, and compounds such as N*- |
| | cyano-N-(tert-pentyl)-N′-3-pyridinyl-guanidine (“P-1075”) as |
| | disclosed in U.S. Pat. No. 5,244,664, which is incorporated |
| | herein by reference; vitamins, such as vitamin E and vitamin C, |
| | and derivatives thereof such as vitamin E acetate and vitamin C |
| | palmitate; hormones, such as erythropoietin, prostaglandins, |
| | such as prostaglandin EI and prostaglandin F2-alpha; fatty |
| | acids, such as oleic acid; diuretics such as spironolactone; heat |
| | shock proteins (“HSP”), such as HSP 27 and HSP 72; calcium |
| | channel blockers, such as Verapamil HCL, Nifedipine, and |
| | diltiazemamiloride; immunosuppressant drugs, such as |
| | Cyclosporine and Fk-506; 5 alpha-reductase inhibitors such as |
| | Finasteride; growth factors such as, EGF, IGF and FGF; |
| | transforming growth factor beta. Preferred hair loss treatment |
| | agents include Minoxidil, 6-(I-piperdinyl)-2,4- |
| | pyrimidinediamine-3-oxide, N′-cyano-N-(tert-pentyl)-N′-3- |
| | pyridinyl-guanidine, finasteride, retinoids and derivatives |
| | thereof, Ketoconazole, Elubiol or mixtures thereof. |
| Inhibition of Hair | Examples of actives suitable for use in inhibiting hair growth |
| Growth | include but not limited to, serine proteases such as trypsin; |
| | vitamins such as alpha-tocopherol (vitamin E) and derivatives |
| | thereof such as Tocopherol acetate and Tocopherol palmitate; |
| | Retinol, Isotretinoin, and betamethoisone. |

### DETAIL DESCRIPTION OF INVENTION

### Example #1

### Topical Anti-aging Moisturizer Composition Composition

PHASE A

| Ingredient | Range (%) |
|---|---|
| Ammonium Lactate | 1-20 |
| Water | Q.S. to 100 |

PHASE B

| | |
|---|---|
| Almond oil | 0.1-20 |
| Avocado oil | 0.1-20 |
| Caprylic/capric triglyceride | 0.1-20 |
| Soybean oil | 0.1-20 |
| Phosphatidyl choline (PC), | 0.01-20 |
| Hydrogenated phosphatidyl choline (HPC) | 0.01-20 |
| Phosphatidyl glycerol (PG) | 0.01-20 |
| Phosphatidyl ethanolamine polyethylene glycol (PE- | 0.01-20 |
| PEG) | |
| Stearyl alcohol | 0.1-10 |
| Menthol | 0.01-1 |

PHASE C

| | |
|---|---|
| Vitamin E | 0.1-1 |
| Sodium ascorbyl phosphate | 0.1-10 |
| α-Bisabolal | 0.1-1 |
| Mixture of Decamethylcyclopentasiloxane and | 1-10 |
| Octamethylcyclotetrasiloxane | |
| Phenoxyethanol (and) Benzoic acid (and) | 0.2-1.2 |
| Dehydroacetic acid | |
| Neem extract | 0.1-10 |
| Sandalwood oil | 0.1-5 |
| Turmeric extract | 0.1 to 10 |
| Basil extract | 0.1-5 |

### Process Description

To a suitable container add ingredients listed in Phase A.

To a separate container add ingredients listed in Phase B.

Heat contents in both containers to 70° C.

While stirring add Phase A to Phase B.

While stirring allow the product to cool down to 35° C.

While stirring add one by one contents of Phase C.

Homogenize with a high shear mixer for 5 minutes.

While stirring at low shear allow product to attain room temperature.

### Example #2

### Skin Moisturizing Composition to Prevent and Treat Dry Skin and Eczema

### Composition:

PHASE A

| Ingredient | Range (%) |
|---|---|
| Water | Q.S. to 100 |

PHASE B

| | |
|---|---|
| Almond oil | 0.1-20 |
| Avocado oil | 0.1-20 |
| Caprylic/capric triglyceride | 0.1-10 |
| Soybean oil | 0.1-10 |
| Phosphatidyl choline (PC), | 0.01-20 |
| Hydrogenated phosphatidyl choline (HPC) | 0.01-20 |
| Phosphatidyl glycerol (PG) | 0.01-20 |
| Phosphatidyl ethanolamine polyethylene glycol (PE- | 0.01-20 |
| PEG) | |
| Stearyl alcohol | 0.1-10 |

PHASE C

| | |
|---|---|
| Vitamin E | 0.1-1 |
| Sodium ascorbyl phosphate | 0.1-10 |
| α-Bisabolal | 0.1-1 |
| Mixture of Decamethylcyclopentasiloxane and | 1-10 |
| Octamethylcyclotetrasiloxane | |
| Phenoxyethanol (and) Benzoic acid (and) | 0.2-1.2 |
| Dehydroacetic acid | |
| Neem extract | 0.01-5 |
| Sandalwood oil | 0.1-5 |
| Turmeric extract | 0.1-10 |
| Aloe vera | 0.1-5 |

### Process Description

To a suitable container add ingredients listed in Phase A.

To a separate container add ingredients listed in Phase B.

Heat contents in both containers to 70° C.

While stirring add Phase A to Phase B.

While stirring allow the product to cool down to 35° C.

While stirring add one by one contents of Phase C.

Homogenize with a high shear mixer for 5 minutes.

Allow product to attain room temperature while stirring at low speed.

### Example #3

### Skin Care Composition with Salicylic Acid to Treat Acne, Psoriasis, Polymyositis, Peri-Vascularitis

### Composition:

PHASE A

| Ingredient | Range (%) |
|---|---|
| Water | Q.S. to 100 |
| Sodium Salicylate | 0.1-5 |

PHASE B

| | |
|---|---|
| Almond oil | 0.1-20 |
| Avocado oil | 0.1-20 |
| Caprylic/capric triglyceride | 0.1-10 |
| Soybean oil | 0.1-10 |
| Phosphatidyl choline (PC), | 0.01-20 |
| Hydrogenated phosphatidyl choline (HPC) | 0.01-20 |
| Phosphatidyl glycerol (PG) | 0.01-20 |
| Phosphatidyl ethanolamine polyethylene glycol (PE- | 0.01-20 |
| PEG) | |
| Stearyl alcohol | 1-10 |

PHASE C

| | |
|---|---|
| Vitamin E | 0.1-1 |
| Sodium ascorbyl phosphate | 0.1-10 |
| α-Bisabolal | 0.1-1 |
| Mixture of Decamethylcyclopentasiloxane and | 1-10 |
| Octamethylcyclotetrasiloxane | |
| Phenoxyethanol (and) Benzoic acid (and) | 0.2-1.2 |
| Dehydroacetic acid | |
| Neem extract | 0.1-5 |
| Sandalwood oil | 0.1-5 |
| Turmeric extract | 0.1 to 10 |
| Aloe vera gel | 0.1-5 |

### Process Description

To a suitable container add ingredients listed in Phase A.

To a separate container add ingredients listed in Phase B.

Heat contents in both containers to 70° C.

While stirring add Phase A to Phase B.

While stirring allow the product to cool down to 35° C.

While stirring add one by one contents of Phase C.

Homogenize with a high shear mixer for 5 minutes.

Allow product to attain room temperature while stirring at low speed.

### Example #4

### Topical Anti-Acne Cream with Benzyl Peroxide

### Composition:

PHASE A

| Ingredient | Range (%) |
|---|---|
| Benzyl Peroxide | 0.1-5 |
| Water | Q.S. to 100 |

PHASE B

| | |
|---|---|
| Almond oil | 0.1-20 |
| Avocado oil | 0.1-20 |
| Caprylic/capric triglyceride | 0.1-10 |
| Soybean oil | 0.1-10 |
| Phosphatidyl choline (PC), | 0.01-20 |
| Hydrogenated phosphatidyl choline (HPC) | 0.01-20 |
| Phosphatidyl glycerol (PG) | 0.01-20 |
| Phosphatidyl ethanolamine polyethylene glycol (PE- | 0.01-20 |
| PEG) | |
| Stearyl alcohol | 1-10 |

PHASE C

| | |
|---|---|
| Vitamin E | 0.1-1 |
| Sodium ascorbyl phosphate | 0.1-10.0 |
| α-Bisabolal | 0.1-1 |
| Mixture of Decamethylcyclopentasiloxane and | 1-10 |
| Octamethylcyclotetrasiloxane | |
| Phenoxyethanol (and) Benzoic acid (and) | 0.2-1.2 |
| Dehydroacetic acid | |
| Neem extract | 0.1-5 |
| Sandalwood oil | 0.1-5 |
| Turmeric extract | 0.1-10 |
| Aloe vera | 0.1-5 |

### Process Description

To a suitable container add ingredients listed in Phase A.

To a separate container add ingredients listed in Phase B.

Heat contents in both containers to 70° C.

While stirring add Phase A to Phase B.

While stirring allow the product to cool down to 35° C.

While stirring add one by one contents of Phase C.

Homogenize with a high shear mixer for 5 minutes.

Allow product to attain room temperature while stirring at low speed.

### Example #5

### Skin Care Composition with Retinol to Treat Acne, Psoriasis, Polymyositis, Peri-Vascularitis

### Composition:

PHASE A

| Ingredient | Range (%) |
|---|---|
| Water | Q.S. to 100 |

PHASE B

| | |
|---|---|
| Almond oil | 0.1-20 |
| Avocado oil | 0.1-20 |
| Caprylic/capric triglyceride | 0.1-10 |
| Soybean oil | 0.1-10 |
| Retinol | 0.01-0.5 |
| Phosphatidyl choline (PC), | 0.01-20 |
| Hydrogenated phosphatidyl choline (HPC) | 0.01-20 |
| Phosphatidyl glycerol (PG) | 0.01-20 |
| Phosphatidyl ethanolamine polyethylene glycol (PE- | 0.01-20 |
| PEG) | |
| Stearyl alcohol | 1-10 |
| Menthol | 0.01-1 |

PHASE C

| | |
|---|---|
| Vitamin E | 0.1-1 |
| Ascorbyl palmitate | 0.1-10 |
| α-Tocopherol | |
| α-Bisabolal | 0.1-1 |
| Mixture of Decamethylcyclopentasiloxane and | 1-10 |
| Octamethylcyclotetrasiloxane | |
| Phenoxyethanol (and) Benzoic acid (and) | 0.2-1.2 |
| Dehydroacetic acid | |
| Neem extract | 0.1 to 10 |
| Sandalwood oil | 0.1-5 |
| Turmeric extract | 0.1 to 10 |

### Process Description

To a suitable container add ingredients listed in Phase A.

To a separate container add ingredients listed in Phase B.

Heat contents in both containers to 70° C.

While stirring add Phase A to Phase B.

While stirring allow the product to cool down to 35° C.

While stirring add one by one contents of Phase C.

Homogenize with a high shear mixer for 5 minutes.

Allow product to attain room temperature while stirring at low speed.

### Example #6

### Topical Eye Lifting Cream Composition with Phenylethyl Resorcinol and Vitamin C

### Composition:

PHASE A

| Ingredient | Range (%) |
|---|---|
| Lactic acid | 0.1-5 |
| Water | Q.S. to 100 |

PHASE B

| | |
|---|---|
| Almond oil | 0.1-20 |
| Avocado oil | 0.1-20 |
| Caprylic/capric triglyceride | 0.1-10 |
| Soybean oil | 0.1-10 |
| Phosphatidyl choline (PC), | 0.01-20 |
| Hydrogenated phosphatidyl choline (HPC) | 0.01-20 |
| Phosphatidyl glycerol (PG) | 0.01-20 |
| Phosphatidyl ethanolamine polyethylene glycol | 0.01-20 |
| (PE-PEG) | |
| Stearyl alcohol | 1-10 |

PHASE C

| | |
|---|---|
| Phenylethyl resorcinol | 0.1-2 |
| Vitamin E | 0.1-1 |
| Sodium ascorbyl phosphate | 0.1-20 |
| α-Bisabolal | 0.1-1 |
| Mixture of Decamethylcyclopentasiloxane and | 1-10 |
| Octamethylcyclotetrasiloxane | |
| Phenoxyethanol (and) Benzoic acid (and) | 0.2-1.2 |
| Dehydroacetic acid | |
| Di Sodium EDTA | 0.1-1 |
| Neem extract | 0.1-5 |
| Sandalwood oil | 0.1-5 |
| Turmeric extract | 0.1-10 |
| Aloe vera | 0.1-5 |
| Fragrance | 0.0-1 |

### Process Description

To a suitable container add ingredients listed in Phase A.

To a separate container add ingredients listed in Phase B.

Heat contents in both containers to 70° C.

While stirring add Phase A to Phase B.

While stirring allow the product to cool down to 35° C.

While stirring add one by one contents of Phase C.

Homogenize with a high shear mixer for 5 minutes.

Allow product to attain room temperature while stirring at low speed.

### Example #9

### Topical Composition to Inhibit Pigment Production in the Skin

### Composition:

PHASE A

| Ingredient | Range (%) |
|---|---|
| Water | Q.S. to 100 |

PHASE B

| | |
|---|---|
| Avocado oil | 0.1-20 |
| Almond oil | 0.1-20 |
| Soybean oil | 0.1-10 |
| Caprylic/capric triglyceride | 0.1-10 |
| Phosphatidyl choline (PC), | 0.01-20 |
| Hydrogenated phosphatidyl choline (HPC) | 0.01-20 |
| Phosphatidyl glycerol (PG) | 0.01-20 |
| Phosphatidyl ethanolamine polyethylene glycol | 0.01-20 |
| (PE-PEG) | |
| Stearyl alcohol | 1-10 |

PHASE C

| | |
|---|---|
| Niacinamide | 0.1-5 |
| Vitamin E | 0.1-1 |
| Sodium ascorbyl phosphate | 0.1-20 |
| α-Bisabolal | 0.1-1 |
| Mixture of Decamethylcyclopentasiloxane and | 1-10 |
| Octamethylcyclotetrasiloxane | |
| Phenoxyethanol (and) Benzoic acid (and) | 0.2-1.2 |
| Dehydroacetic acid | |
| Di Sodium EDTA | 0.1-1 |
| Neem extract | 0.1-5 |
| Sandalwood oil | 0.1-5 |
| Turmeric extract | 0.1-10 |
| Aloe vera | 0.1-5 |
| Fragrance | 0.0-1 |

### Process Description

To a suitable container add ingredients listed in Phase A.

To a separate container add ingredients listed in Phase B.

Heat contents in both containers to 70° C.

While stirring add Phase A to Phase B.

While stirring allow the product to cool down to 35° C.

While stirring add one by one contents of Phase C.

Homogenize with a high shear mixer for 5 minutes.

Allow product to attain room temperature while stirring at low speed.

### Example #8

### Topical Anti-Fungal Cream with Terbinafine Hydrochloride to Treat Fungal Infections of the Toenail and Fingernail

### Composition:

PHASE A

| Ingredient | Range (%) |
|---|---|
| Terbinafine hydrochloride | 0.1-5 |
| Water | Q.S. to 100 |

PHASE B

| | |
|---|---|
| Avocado oil | 0.1-20 |
| Almond oil | 0.1-20 |
| Soybean oil | 0.1-10 |
| Caprylic/capric triglyceride | 0.1-10 |
| Phosphatidyl choline (PC) | 0.01-20 |
| Hydrogenated phosphatidyl choline (HPC) | 0.01-20 |
| Phosphatidyl glycerol (PG) | 0.01-20 |
| Phosphatidyl ethanolamine polyethylene glycol | 0.01-20 |
| (PE-PEG) | |
| Stearyl alcohol | 1-10 |

PHASE C

| | |
|---|---|
| Vitamin E | 0.1-1 |
| Sodium ascorbyl phosphate | 0.1-20 |
| α-Bisabolal | 0.1-1 |
| Mixture of Decamethylcyclopentasiloxane and | 1-10 |
| Octamethylcyclotetrasiloxane | |
| Phenoxyethanol (and) Benzoic acid (and) | 0.2-1.2 |
| Dehydroacetic acid | |
| Di Sodium EDTA | 0.1-1 |
| Neem extract | 0.1-5 |
| Sandalwood oil | 0.1-5 |
| Turmeric extract | 0.1-10 |
| Aloe vera | 0.1-5 |
| Fragrance | 0.0-0.5 |

### Process Description

To a suitable container add ingredients listed in Phase A.

To a separate container add ingredients listed in Phase B.

Heat contents in both containers to 70° C.

While stirring add Phase A to Phase B.

While stirring allow the product to cool down to 35° C.

While stirring add one by one contents of Phase C.

Homogenize with a high shear mixer for 5 minutes.

Allow product to attain room temperature while stirring at low speed.

### Example #9

### Topical Composition for Hair Growth with 5% Minoxidil

### Composition:

PHASE A

| Ingredient | Range (%) |
|---|---|
| Minoxidil | 1-10 |
| Propylene glycol | 1-10 |
| Ethanol | 0.1-2 |
| Water | Q.S. to 100 |

PHASE B

| | |
|---|---|
| Avocado oil | 0.1-20 |
| Almond oil | 0.1-20 |
| Soybean oil | 0.1-10 |
| Caprylic/capric triglyceride | 0.1-10 |
| Phosphatidyl choline (PC), | 0.01-20 |
| Hydrogenated phosphatidyl choline (HPC) | 0.01-20 |
| Phosphatidyl glycerol (PG) | 0.01-20 |
| Phosphatidyl ethanolamine polyethylene glycol | 0.01-20 |
| (PE-PEG) | |
| Stearyl alcohol | 1-10 |

PHASE C

| | |
|---|---|
| Diethyl Isosorbide | 0.1-10 |
| Vitamin E | 0.1-1 |
| Sodium ascorbyl phosphate | 0.1-10 |
| α-Bisabolal | 0.1-1 |
| Mixture of Decamethylcyclopentasiloxane and | 1-10 |
| Octamethylcyclotetrasiloxane | |
| Phenoxyethanol (and) Benzoic acid (and) | 0.2-1.2 |
| Dehydroacetic acid | |
| Neem extract | 0.1-5 |
| Sandalwood oil | 0.1-5 |
| Turmeric extract | 0.1 to 10 |
| Aloe vera | 0.1-5 |
| Fragrance | 0.0-0.5 |

### Process Description

To a suitable container add ingredients listed in Phase A.

To a separate container add ingredients listed in Phase B.

Heat contents in both containers to 70° C.

While stirring add Phase A to Phase B.

While stirring allow the product to cool down to 35° C.

While stirring add one by one contents of Phase C.

Homogenize with a high shear mixer for 5 minutes.

Allow product to attain room temperature while stirring at low speed.

### Example #10

### Topical Composition for Nonsteroidal Anti-Inflammatory Drug (NSAID) to Relieve Pain in the Joints and Muscles

### Composition:

PHASE A

| Ingredient | Range (%) |
|---|---|
| Water | Q.S. to 100 |

PHASE B

| | |
|---|---|
| Avocado oil | 0.1-20 |
| Almond oil | 0.1-20 |
| Soybean oil | 0.1-10 |
| Caprylic/capric triglyceride | 0.1-10 |
| Phosphatidyl choline (PC), | 0.01-20 |
| Hydrogenated phosphatidyl choline (HPC) | 0.01-20 |
| Phosphatidyl glycerol (PG) | 0.01-20 |
| Phosphatidyl ethanolamine polyethylene glycol | 0.01-20 |
| (PE-PEG) | |
| Stearyl alcohol | 1-10 |

PHASE C

| | |
|---|---|
| Diclofenac sodium | 0.1-5 |
| Diethyl isosorbide | 0.1-10 |
| Vitamin E | 0.1-1 |
| Sodium ascorbyl phosphate | 0.1-20 |
| α-Bisabolal | 0.1-1 |
| Mixture of Decamethylcyclopentasiloxane and | 1-10 |
| Octamethylcyclotetrasiloxane | |
| Phenoxyethanol (and) Benzoic acid (and) | 0.2-1.2 |
| Dehydroacetic acid | |
| Di Sodium EDTA | 0.1-1 |
| Neem extract | 0.1-5 |
| Sandalwood oil | 0.1-5 |
| Turmeric extract | 0.1-10 |
| Aloe vera | 0.1-5 |
| Fragrance | 0.0-1 |

### Process Description

To a suitable container add ingredients listed in Phase A.

To a separate container add ingredients listed in Phase B.

Heat contents in both containers to 70° C.

While stirring add Phase A to Phase B.

While stirring allow the product to cool down to 35° C.

While stirring add one by one contents of Phase C.

Homogenize with a high shear mixer for 5 minutes.

Allow product to attain room temperature while stirring at low speed.

### REFERENCES

1. U.S. Pat. No. 6,623,753 Sep. 23, 2003 Bodmer et al Allylamine containing liposomes,
2. Enhanced delivery of retinoic acid to skin by cationic liposomes. Kitagawa S, Kasamaki M. Chem Pharm Bull (Tokyo). 2006 February; 54(2): 242-4.
3. Lecithin organogels as a potential phospholipid-structured system for topical drug delivery: a review. Kumar R, Katare O P. AAPS PharmSciTech. 2005 Oct. 6; 6(2):E298-310.
4. In vivo study of liposomes as drug carriers to oral mucosa using EPR oximetry. Erjavec V, Pavlica Z, Sentjurc M, Petelin M. Int J. Pharm. 2006 Jan. 3; 307(1):1-8. Epub 2005 Oct. 27.
5. Liposomes and niosomes as topical drug delivery systems. Choi M J, Maibach H I. Skin Pharmacol Physiol. 2005 Sep.-Oct.; 18(5):209-19. Review.
6. Ethosomes for skin delivery of ammonium glycyrrhizinate: in vitro percutaneous permeation through human skin and in vivo anti-inflammatory activity on human volunteers.
Paolino D, Lucania G, Mardente D, Alhaique F, Fresta. J Control Release. 2005 Aug. 18; 106(1-2):99-110.
7. Pilot study of topical delivery of methotrexate by electroporation.
Wong T W, Zhao Y L, Sen A, Hui S W. Br J. Dermatol. 2005 March; 152(3):524-30.
8. Evaluation of in-vivo topical anti-inflammatory activity of indometacin from liposomal vesicles
Puglia C, Trombetta D, Venuti V, Saija A, Bonina F. J Pharm Pharmacol. 2004 October; 56(10):1225-32.
9. Effect of edge activators on the formation and transfection efficiency of ultradeformable liposomes.
Lee E H, Kim A, Oh Y K, Kim C K. Biomaterials. 2005 Jan.; 26(2):205-10.
10. Hydroxyzine from topical phospholipid liposomal formulations: evaluation of peripheral antihistaminic activity and systemic absorption in a rabbit model.
Elzainy A A, Gu X, Simons F E, Simons K J. AAPS Pharm Sci. 2003 Nov. 5; 5(4):E28.
11. Iontophoresis of lecithin vesicles of cyclosporin A.
Boinpally R R, Zhou S L, Devraj G, Anne P K, Poondru S, Jasti BR. Int J. Pharm. 2004 Apr. 15; 274(1-2):185-90.
12. Enhanced transfollicular delivery of adriamycin with a liposome and iontophoresis.
Han I, Kim M, Kim J. Exp Dermatol. 2004 February; 13(2):86-92.
13. Lecithin vesicles for topical delivery of diclofenac.
Boinpally R R, Zhou S L, Poondru S, Devraj G, Jasti B R.
Eur J Pharm Biopharm. 2003 November; 56(3):389-92.
14. Vehicle effects on in vitro skin permeation of thiocolchicoside. Bonina F, Puglia C, Trombetta D, Dragani M C, Gentile M M, Clavenna G. Pharmazie. 2002 November; 57(11):750-2.
15. Transdermal delivery of cyclosporin A solubilized in mixed micelles through mice skin] Wu T, Guo J X, Ping Q N, Jin F Y, Sun X W. Yao Xue Xue Bao. 2001 May; 36(5):381-5. Chinese.
16. Paolino D, Ventura C A, Nistico S, Puglisi G, Fresta Int J. Pharm. 2002 Sep. 5; 244(1-2):21-31.
17. Marinosomes, marine lipid-based liposomes: physical characterization and potential application in cosmetics.
Moussaoui N, Cansell M, Denizot A. Int J. Pharm. 2002 Aug. 21; 242(1-2):361-5.
18. Targeted gene delivery to skin cells in vivo: a comparative study of liposomes and polymers as delivery vehicles.
Raghavachari N, Fahl W E. J Pharm Sci. 2002 March; 91(3):615-22.
19. Interaction of liposome formulations with human skin in vitro.
Betz G, Imboden R, Imanidis G. Int J. Pharm. 2001 Oct. 23; 229(1-2):117-29. Erratum in: Int J Pharm 2002 Jan. 31; 232(1-2):243-4.
20. Enhancement of the antiparakeratotic potency of calcitriol and tacalcitol in liposomal preparations in the mouse tail test.
Korbel J N, Sebok B, Kerenyi M, Mahrle G. Skin Pharmacol Appl Skin Physiol. 2001 Sep.-Oct.; 14(5): 291-5.
21. Enhancement of topical delivery of a lipophilic drug from charged multilamellar liposomes.
Katahira N, Murakami T, Kugai S, Yata N, Takano J Drug Target 1999; 6(6):405-14.
22. A pilot study to evaluate the treatment of basal cell carcinoma with 5-fluorouracil using phosphatidyl choline as a transepidermal carrier. Romagosa R, Saap L, Givens M, Salvarrey A, He J L, Hsia S L, Taylor Dermatol Surg. 2000 Apr.; 26(4):338-40.
23. Application of membrane-based dendrimer/DNA complexes for solid phase transfection in vitro and in vivo.
Bielinska A U, Yen A, Wu H L, Zahos K M, Sun R, Weiner N D, Baker JR Jr, Roessler B J. Biomaterials. 2000 May; 21(9):877-87.
24. Effects of lipid composition on the transcorneal penetration of liposomes containing disulfiram, a potential anti-cataract agent, in the rabbit.
Ito Y, Cai H, Koizumi Y, Hon R, Terao M, Kimura T, Takagi S, Tomohiro Biol Pharm Bull. 2000 March; 23(3):327-33.
25. Prolonged effect of liposomes encapsulating pilocarpine HCl in normal and glaucomatous rabbits.
Monem A S, Ali F M, Ismail M W. Int J. Pharm. 2000 Mar. 30; 198(1):29-38.

26. Lecithin vesicular carriers for transdermal delivery of cyclosporin A.
Guo J, Ping Q, Sun G, Jiao C. Int J. Pharm. 2000 Jan. 25; 194(2):201-7.
27. Topical medications for orofacial neuropathic pain: a review. Padilla M, Clark G T, Merrill R L. J Am Dent Assoc. 2000 February; 131(2):184-95. Review.
28. Study on glycolic acid delivery by liposomes and microspheres.
Perugini P, Genta I, Pavanetto F, Conti B, Scalia S, Baruffini A. Int J. Pharm. 2000 Feb. 25; 196(1):51-61.
29. Influence of lecithin on some physical chemical properties of poloxamer gels: rheological, microscopic and in vitro permeation studies.
Bentley M V, Marchetti J M, Ricardo N, Ali-Abi Z, Collett J H. Int J. Pharm. 1999 Dec. 20; 193(1):49-55.
30. Free versus liposome-encapsulated lignocaine hydrochloride topical applications.
el-Ridy M S, Khalil R M. Pharmazie. 1999 September; 54(9):682-4.
31. Encapsulation and stability of clofazimine liposomes. Patel V B, Misra A N. J. Microencapsul. 1999 May-Jun.; 16(3):357-67.
32. Liposomes as a topical delivery system: the role of size on transport studied by the EPR imaging method.
Sentjurc M, Vrhovnik K, Kristl J. J Control Release. 1999 May 1; 59(1):87-97.
33. Contact hypersensitivity: a simple model for the characterization of disease-site targeting by liposomes.
Klimuk S K, Semple S C, Scherrer P, Hope M J. Biochim Biophys Acta. 1999 Mar. 4; 1417(2):191-201.
34. Phospholipids affect stratum corneum lipid bilayer fluidity and drug partitioning into the bilayers.
Kirjavainen M, Monkkonen J, Saukkosaari M, Valjakka-Koskela R, Kiesvaara J, Urtti A. J Control Release. 1999 Mar. 29; 58(2):207-14.
35. Effect of vehicle on topical liposomal drug delivery: petrolatum bases.
Foldvari M Microencapsul. 1996 Sep.-Oct.; 13(5):589-600.
36. Enhanced transdermal delivery of diazepam by submicron emulsion (SME) creams.
Schwarz J S, Weisspapir M R, Friedman D I. Pharm Res. 1995 May; 12(5):687-92.
37. Molecular and cellular studies of hyaluronic acid-modified liposomes as bioadhesive carriers for topical drug delivery in wound healing. Yerushalmi N, Arad A, Margalit R. Arch Biochem Biophys. 1994 ep; 313(2):267-73.
38. Topical treatment of acute hindlimb lymphedema of the rat using a troxerutin-phosphatidylcholine complex in liposomal-like microdispersion.
Casley-Smith J R, Casley-Smith J R, Curri S, Foldi M. Lymphology. 1993 Mar.; 26(1):25-7.
39. Anti-inflammatory activity of hamamelis distillate applied topically to the skin. Influence of vehicle and dose.
Korting H C, Schafer-Korting M, Hart H, Laux P, Schmid M. Eur J Clin Pharmacol. 1993; 44(4):315-8.
40. Small reduction of capsaicin-induced neurogenic inflammation in human forearm skin by the glucocorticoid prednicarbate.
Teter R, Herbert M K, Schmidt R F, Weis K H. Agents Actions. 1993; 38 Spec No:C31-4.
41. Liposomes from soya phospholipids as percutaneous drug carriers. 1st communication: qualitative in vivo investigations with antibody-loaded liposomes.
Artmann C, Roding J, Ghyczy M, Pratzel H G. Arzneimittelforschung. 1990 December; 40(12):1363-5.
42. Liposomes from soya phospholipids as percutaneous drug carriers. 2nd communication: quantitative in vivo investigations with radioactively labelled liposomes.
Artmann C, Roding J, Ghyczy M, Pratzel H G. Arzneimittelforschung. 1990 December; 40(12):1365-8.
43. Retention of topical liposomal formulations on the cornea.
McCalden T A, Levy M. Experientia. 1990 Jul. 15; 46(7):713-5. PMID: 2373198 [PubMed—indexed for MEDLINE]
44. Effect of liposomes on hamster oral mucosa.
J Biomed Mater Res. 1989 Oct.; 23(10):1213-29.
45. Penetration of lecithin from hydrocortisone-containing liposomes into human skin.
Wohlrab W, Lachmann U, Lasch J. Dermatol Monatsschr. 1989; 175(6):344-7.

What is claimed is:

1. Novel compositions for topical drug delivery of the pharmaceutically active compounds which allow active compounds to pass through the stratum corneum, epidermis and deliver to the deeper layers of the skin, dermis, are developed. The active compounds are encapsulated into the phospholipid spheres or bilayers. The phospholipid emulsion technology utilized in the skincare products provides a vastly superior method of topical delivery of the active compounds. This method allows pharmacologically active compounds to penetrate through outer layers of the skin to reach the deep layers. As a result, the products are highly effective, cosmetically elegant and gentle on the skin.

2. The composition of claim 1 comprising one or more oils including but not limited to Almond oil, Avocado oil, Caprylic/capric triglyceride, Corn oil, Cottonseed oil, Evening primrose oil, Olive oil, Peanut oil, Safflower oil, Sesame oil, Soybean oil, Wheat germ oil.

3. The composition of claim 2 comprising one or more of the following phospholipids. In this technology phospholipids play several roles.
Stabilize oil in water emulsion
Encapsulate pharmacologically active compound in its bilayer structure
Carries active compound underneath the stratum cornium and deliver it to the deeper layers of the skin, Epidermis and Dermis
The following phospholipids alone or in combination with other phospholipids may be used. Various lipids include Dimyristoyl-phosphatidyl-choline (DMPC), Dimyristoyl-phosphatidyl-glycerol (DMPG), Dioleoyl-phosphatidyl-choline (DOPC) Dioleoyl-phosphatidyl-glycerol (DOPG), Dioleoyl-phosphatidyl-serine (DOPS), Dipalmitoyl-phosphatidyl-choline (DPPC), Distearoyl-phosphatidyl-choline (DSPC), Distearoyl-phosphatidyl-glycerol (DSPG), Hydrogenated phosphatidyl choline (HPC), Phosphatidyl-choline (PC), Phosphatidylethanolaminepolyethyleneglycol (PE-PEG), Phophatidyl-glycerol (PG), Palmitoyl-oleoyl-phosphatidyl-choline (POPC), Palmitoyl-oleoyl-phosphatidyl-glycerol (POPG), Phosphatidyl-serine (PS).

4. Composition of claim 3 comprising a suitable amount of water.

5. The pharmacological active compound from Table 1 may be incorporated in the aqueous or nonaqueous phase depending on its solubility.

6. Composition of claim 3 comprising one or more fatty alcohols including but not limited to Cetyl Alcohol, Stearyl alcohol, Cetostearyl alcohol. These alcohols act as emulsion stabilizer, co-emulsifier and thickener.

7. Composition of claim 4 comprising one or more anti-oxidant including but not limited to Vitamin C, Vitamin E or Butylated hydroxy toluene.

8. Composition of claim 4 comprising of one or more anti-skin irritants including but not limited to α—Bisabolal (natural or synthetic), Aloe vera, Panthenol, and Chamomile.

9. Composition of claim 4 comprising one or more moisturizers including but not limited to silicone fluid, Cyclopenta siloxane, Cyclomethicone, Dimethicone copolyol, Dow Corning 1401, 200, 1501 fluids.

10. Composition of claim 4 comprising of Menthyl lactate, Menthol or its salt form.

11. Composition of claim 4 comprising commonly used preservatives in skin care formulations including but not limited to combination of Methyl paraben and Propyl paraben, Diazolidinyl urea, Benzyl alcohol, disodium EDTA, para Hydrobenzoic acid, Phenoxyethanol, Benzoic acid, Dehydroacetic acid, Caprylyl glycol, Sorbic acid.

12. Composition of claim 4 comprising a drug absorption enhancer including but not limited to Oleic acid, Oleyl alcohol, Avocado oil or Ethanol.

13. Composition of claim 4 comprising of essential oil extracts including Sandalwood oil, Jasmine oil, and Rose oil.

14. Composition of claim 4 comprising of Ayurvedic actives including Sandalwood, Turmeric, Neem, and Basil.

15. Composition of claim 4 comprising a suitable fragrance.
